(19) United States
(12) Patent Application Publication
(10) Pub. No.: US 2026/0325643 A1
(43) Pub. Date: Oct. 22, 2026

(54) METHOD AND PHARMACEUTICAL COMPOSITION FOR VISIBLE LIGHT-INDUCED CORNEAL CROSSLINKING WITH RUTHENIUM COMPOUNDS FOR THE TREATMENT OF EYE DISORDERS

(71) Applicant: KOC UNIVERSITESI, Istanbul (TR)

(72) Inventors: Seda KIZILEL, Istanbul (TR); Afsun SAHIN, Istanbul (TR); Ayse YILDIZ, Istanbul (TR); Muhammad Anwaar NAZEER, Istanbul (TR); Ayesha GULZAR, Istanbul (TR); Erdost YILDIZ, Istanbul (TR)

(73) Assignee: KOC UNIVERSITESI, Istanbul (TR)

(21) Appl. No.: 19/109,576

(22) PCT Filed: Sep. 7, 2022
(86) PCT No.: PCT/TR2022/050954

§ 371 Date: Mar. 7, 2025

(51) Int. Cl. A61K 41/00; A61K 9/00; A61K 9/08; A61K 45/06

(52) U.S. Cl. CPC A61K 41/0057; A61K 9/0048; A61K 9/08; A61K 45/06

(57) ABSTRACT

A method for the photo-crosslinking of corneal collagen under visible light and a pharmaceutical composition including an FDA-approved organoruthenium compound-based photoinitiator are provided. The method includes: applying an analgesic solution to a human eye surface and removing an epithelium layer, applying a photoinitiator pharmaceutical composition including a ruthenium compound on a central corneal surface and waiting for 5 min for an absorption, and exposing a cornea to 3 mW/cm² of the visible light to induce a crosslinking for 40 min. More particularly, the method and pharmaceutical composition described herein are for the treatment of corneal ectatic disorders.

### CROSS REFERENCE TO THE RELATED APPLICATIONS

This application is the national phase entry of International Application No. PCT/TR2022/050954, filed on Sep. 7, 2022, the entire contents of which are incorporated herein by reference.

### TECHNICAL FIELD

The present invention discloses and claims a method for the photo-crosslinking of corneal collagen under visible light and a pharmaceutical composition including an FDA-approved organoruthenium compound-based photo-initiator. More particularly, the method and pharmaceutical composition described herein are for the treatment of corneal ectatic disorders.

### BACKGROUND

Corneal ectatic disorders (Ectatic corneal diseases (ECDs)) are a group of disorders characterized by corneal weakness and irregular remodeling of corneal structure, such as thinning, protrusion, and irregular astigmatism [1].

Keratoconus, one of the most common corneal ectatic disorders, is a clinical problem where clear, dome-shaped cornea tends to thin and bulge outside to form a cone-like shape. This distorted shape disrupts the light passage coming in, and this light deflection from its path leads towards a distorted vision. Corneal tissue crosslinking is performed to retard the progression of keratoconus [2].

Clinical procedure curing keratoconus involves the removal or scraping of epithelial layer exposing stroma. A saline solution containing riboflavin (RF) is applied topically, and simultaneously the eye surface is exposed to UV light (365 nm). Perhaps collagen molecules in the stroma are cross-linked under this treatment, but the exact mechanism is partially unknown. The enhanced corneal rigidity prevents stroma thinning further associated with keratoconus. The RF-catalyzed reaction involves the generation of singlet oxygen, which reacts with available molecules, collagen, and proteoglycans in the stroma [3].

The conventional procedure in the clinics for corneal collagen photo-crosslinking includes the use of UV-A light and riboflavin as the photoinitiator molecule. This procedure is painful, long-lasting, has many side effects, and leads to persistent corneal haze due to corneal toxicity. There are also other pharmacological corneal collagen crosslinking treatment methods, such as copper sulfate eye drops; however, none of them are as effective as corneal collagen photo-crosslinking approach [4].

A method developed by (Ciolino, 2015 [5]; also published in WO2015130944A1) involves Verteporfin/Nonthermal laser cross-linking of the cornea. Verteporfin/non-thermal laser increases covalent bonds through free oxygen radicals. However, there is no study data available with application in thin corneas. In addition, there is not enough information about endothelial toxicity and corneal haze complications. Another method is the Rose Bengal/Green Light (532 nm) cross-linking method, where Rose Bengal is used as a photoinitiator [6]. An increased hardness in the area where cross-linking with Rose Bengal is triggered has been shown. However, a more superficial effect has also been reported, raising the possibility of insufficient results in disease progression and clinical success [7]. In another study [8] is related to use of riboflavin as a photo-initiator. Crosslinking occurs under UV-A as a result of new covalent bond formation between corneal collagen fibrils. This results in increased corneal stiffness. However, the use of UV-A light may result in endothelial toxicity and corneal edema [9].

The standard protocol for corneal crosslinking can only be applied in cases with a corneal thickness above 400 μm [10]. When applied to thinner corneas, UV-A beam reaches the corneal endothelial cells resulting in endothelial toxicity [11] and corneal edema [12]. When such a complication is encountered, bullous keratopathy develops, and the need for keratoplasty arises [13]. Another severe complication associated with this method is keratocyte apoptosis caused by the riboflavin/UV-A effect and comeal clouding (corneal haze) caused by intense apoptosis. While corneal haze heals in 6-12 months in some eyes, it does not improve in some eyes and results in permanent loss of visual acuity [14].

In the International patent document WO2017095240A1, it is mentioned that ruthenium and sodium persulfate (SPS) compounds are used together as a photoinitiator for the photopolymerization process. The wavelength range used is between 400-700 nm, more preferably 400-450 nm. It has been stated that the method using visible light and a ruthenium-SPS mixture has less phototoxicity in cells and can be used in tissue engineering. In the aforementioned document, it is stated that polymers can be synthetic or natural. Collagen is shown as an example of natural polymers. In this patent there is no explanation for biomedical or ophthalmological applications and the mentioned material as an ophthalmological solution. Moreover, there is no experimental data and detailed explanation of the toxic, carcinogenic and/or molecular effects on human cells.

Accordingly, there has been and still is a need to design a new method that overcomes the technical issues mentioned above. These needs and other needs are satisfied by the present invention.

The present invention relates to photo-crosslinking of corneal collagen under visible light through the use of an FDA-approved organoruthenium compound-based photoinitiator. The combination of ruthenium along with the use of visible light will prevent UV-A light-induced cytotoxicity on human cornea and hence will be important for patient compliance, improve the comfort and will lead to the development of a clinically applicable high-quality procedure.

### SUMMARY

According to a first aspect of the invention there is provided a method for the photo-crosslinking of corneal collagen under visible light through the use of an FDA-approved organoruthenium compound-based photoinitiator.

Another aspect of the present invention relates to a pharmaceutical composition including ruthenium, an FDA-approved safe photo-initiator, as a new cross-linking agent.

The present invention aims at getting rid of the negative effects of the ruthenium cross-linking process by using visible light instead of UV-A light. Ruthenium and visible blue light combination is also designed to shorten the duration of the standard procedure and its negative effects.

The present invention is also aimed to increase the biomechanical strength of corneal stroma for the treatment of corneal ectatic diseases, such as keratoconus with the help of visible light and ruthenium as a photoinitiator. With this approach through the use of visible light and pharmaceutically acceptable and biocompatible ruthenium, corneal collagen photo-crosslinking therapy can be applied to all corneal ectatic patients and there will be significantly lower toxicity and lower number of complications.

In a further aspect, the present invention relates to a method provided of applying a pharmaceutical composition to a subject (human, non-human individual, object or surface area) and a pharmaceutical composition of administering it to a subject is provided.

Another embodiment of the present invention relates to a method and a pharmaceutical composition for the treatment of corneal ectatic disorders such as keratoconus. The invention can be used for the preparation and application of an ophthalmological photo therapy useful in the treatment and/or prevention of corneal ectatic disorders.

Yet another objective of the present invention is to provide a pharmaceutical composition including a pharmaceutical carrier and a therapeutically effective amount of ruthenium.

Accordingly, a broad embodiment of the invention is directed to a photo therapy method which can be used for biomedical applications.

As a novel biomedical application method, clinical therapy options with photodynamic therapies are exponentially increases every year. Due to the growing field of tissue engineering-based medicine, this visible light-based corneal crosslinking method will have one major clinical application.

As a major clinical application, ruthenium based visible light corneal collagen photo-crosslinking method will be used to non-surgical therapy of corneal ectatic diseases, such as keratoconus, keratoglobus, and post-surgery ectasia. This method will make it possible to cure all patient populations with higher efficiency and significantly lower complication rate.

This object and other objects of this invention become apparent from the detailed discussion of the invention that follows.

### BRIEF DESCRIPTION OF THE DRAWINGS

The present invention is illustrated in the accompanying figures wherein;

FIG. 1 is a schematic of key steps for corneal crosslinking with Ruthenium and visible light.

FIGS. 2A-2B Representation of collagen photo-crosslinking mechanism of ruthenium and sodium persulfate compounds. (FIG. 2A) Under visible light (430 nm) illumination, Ru(II) and sodium persulfate (SPS) generate Ru(III), SPS anions, and reactive oxygen species. (FIG. 2B) Photo-crosslinking of tyrosine groups on corneal collagen structures with Ruthenium/visible light-based CXL procedure.

FIGS. 3A-3B are illustrations of evaluation of the effect of ruthenium-mediated crosslinking treatment on viability of different cell types: (FIG. 3A) Human Corneal Epithelial Cells, and (FIG. 3B) Limbal Mesenchymal Stem Cells. The half-maximum inhibitory concentration (IC50) of [Ru(bpy)₃]²⁺ plus SPS treatment was found as 1.162 mM for human corneal epithelial cells, and 2.732 mM for limbal mesenchymal stem cells. It can be seen that the [Ru(bpy)₃]²⁺-SPS solution has significantly high cell viability (90%) than separate compounds of [Ru(bpy)₃]²⁺ and SPS mixture.

FIGS. 4A-4H Flow cytometry measurements with Annexin V-FITC and PI to observe apoptosis levels in LMSCs and HCECs. (FIGS. 4A-4D) Flow cytometry results and corresponding quantitate analysis for LMSCs and (FIGS. 4E-4H) flow cytometry results and corresponding quantitate analysis for HCECs after 10 mM and 1 mM [Ru(bpy)3]2+-SPS treatments.

FIGS. 5A-5B are two illustrations of hybrid rheometer results for control, riboflavin, and ruthenium induced crosslinked corneas. Two different corneal crosslinking application period could be seen: (FIG. 5A) 10 min and (FIG. 5B) 40 min. This result indicates that photo-crosslinking effect of the ruthenium compound is concentration-dependent. And the 1 mM concentration of [Ru(bpy)₃]²⁺-SPS solution indicates the best crosslinking efficiency.

FIGS. 6A-6D (FIG. 6A) Uniaxial stress-strain test results for control and crosslinked corneas. (FIG. 6B) Osmotic stress for control and crosslinked corneas. (FIG. 6C) Optical transparency measurements of control and crosslinked comeas. (FIG. 6D) Enzymatic digestion test results for control and crosslinked corneas. Statistical significance of results indicated with asterisks: * p<0.05, ** p<0.01, and *** p<0.001.

FIGS. 7A-7B Optical property analysis of control, riboflavin, and ruthenium based crosslinked bovine corneas in PBS in both UV (300-400) and visible (400-800) via UV & visible light spectrophotometer. Optical transmission (FIG. 7A) and optical absorbance (FIG. 7B) of the corneas did not change significantly after different crosslinking procedure applications.

### DETAILED DESCRIPTION OF THE EMBODIMENTS

The present invention describes a novel method for the photo-crosslinking of corneal collagen under visible light through the use of an FDA-approved organoruthenium compound-based photoinitiator.

The present invention is to be used in the treatment of keratoconus patients by converting ruthenium, a safe FDA-approved photo-initiator, based [Ru(bpy)₃] into a new cross-linking agent. It is aimed to cause less toxicity and less complications compared to similar methods applied in the treatment of corneal ectatic patients.

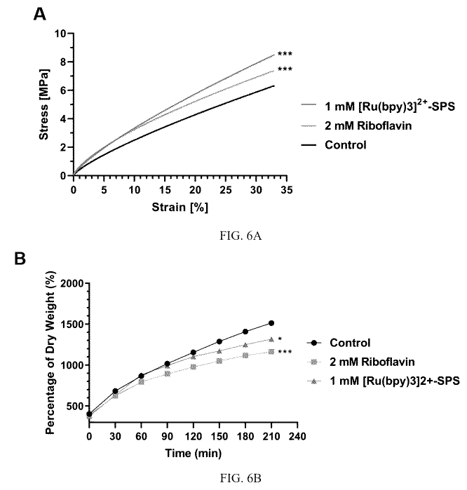

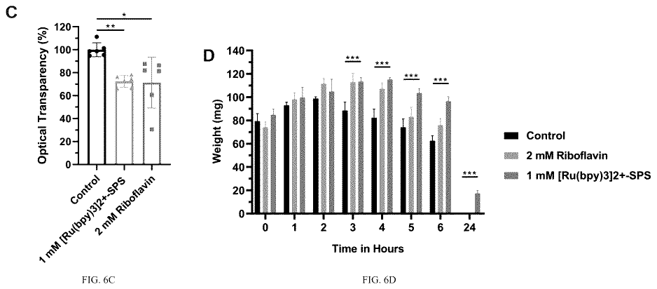

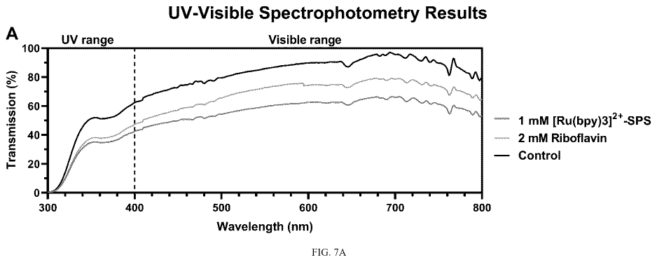

The standard protocol for corneal crosslinking can only be applied in cases with a corneal thickness above 400 μm. When applied to thinner corneas, UV-A beam reaches the corneal endothelial cells resulting in endothelial toxicity and corneal edema. For this purpose, the Lambert-Beer formula was applied for optical transmittance results of both photoinitiator solutions (FIG. 6C). Corneal light transmittance was then calculated with the Lambert-Beer formula:

A=ε×c×l

In this formula, A represents the optical absorbance of the sample, ε represents the molar attenuation coefficient of the photoinitiator, c is the concentration of the photoinitiator, and l is the total optical path length of the light. For these calculations, molar attenuation coefficients for each photoinitiators (10066 M⁻¹ cm⁻¹ for riboflavin and 14,600 M⁻¹ cm⁻¹ for tris(2,2′-bipyridyl)ruthenium(II)) obtained from previous literature.[15, 16] While the mean distance that riboflavin traveled in the bovine cornea is 719 μm, the mean distance for ruthenium solution is 575 μm. This result shows that ruthenium will reach fewer toxic levels in the inner parts of the cornea, which enables lower corneal endothelial toxicity for further clinical applications. According to the results, it is found that the method is also suitable for use in corneas thinner than 400 μm, because neither ruthenium-based solution nor visible light did not reach toxic levels in 400 μm.

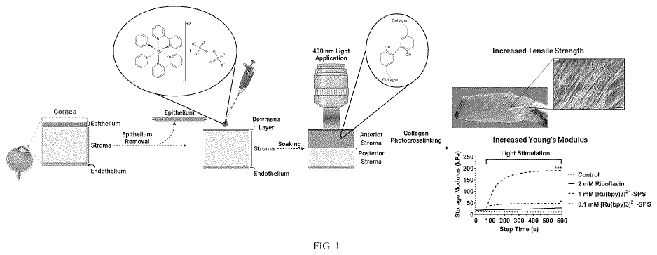

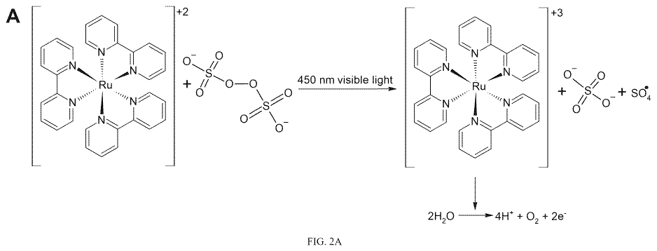

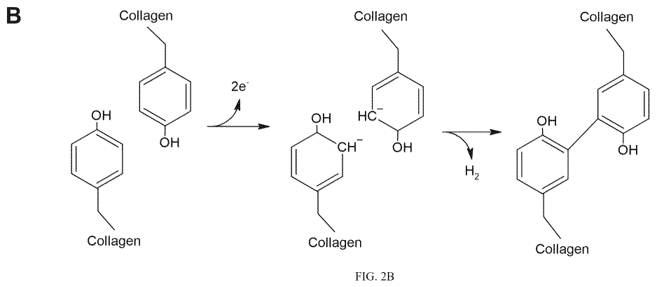

According to the invention, a method for the photo-crosslinking of corneal collagen under visible light including the step of (FIG. 1):
applying an analgesic solution to a human eye surface and removing the epithelium layer,
applying a pharmaceutical composition including ruthenium on the central corneal surface and waiting for 5 min for absorption,
exposing the cornea to visible light to induce crosslinking for 10 to 20 min.

According to the present invention, the first step includes a preparation step before the procedure. An analgesic eye drop is applied to the cornea and the epithelium is removed with an ophthalmic scalpel.

In the second step, a solution includes ruthenium are dropped on the central corneal surface and waited for 5 min for absorption.

In the third step, the corneas are then exposed to 3 m W/cm² visible light (430 nm-450 nm, or 430 nm, or 450 nm) for 10 to 40 min and after this process the ophthalmic gel is applied to prevent the corneas from drying out.

In one embodiment of the invention, the visible light is at a wavelength range of 400-450 nm. Narrow-spectrum blue light is a type of high energy visible light, also defined as having a wavelength between 400 and 450 nm. More preferably, the visible light is a blue visible light at 430-450 nm wavelengths.

As used herein, the compound name “ruthenium” refers tris(bipyridine)ruthenium(II) with the formula [Ru(bpy)₃]²⁺. It is a ruthenium (Ru) based transition metal complex. 14. According to the present invention the ruthenium compound is used in the treatment and/or prevention of corneal ectatic disorder.

For use in the treatment and/or prevention of corneal ectatic disorder, a photo-initiator pharmaceutical composition includes:
a therapeutically effective amount of ruthenium compound and sodium persulfate; and
a pharmaceutically acceptable carrier.

According to the invention, the pharmaceutical composition includes ruthenium compound. Furthermore, the composition includes sodium persulfate (SPS). Preferably, the photo-initiator is a combination of ruthenium compound and sodium persulfate. The composition is a solution for ophthalmic applications. More preferably, the pharmaceutical composition is an ophthalmic solution administered topically as an eye drop.

Unless specified otherwise, the term “ruthenium solution” refers to the composition includes ruthenium and sodium persulfate (SPS). Usage of ruthenium based organoruthenium complexes as photoinitiator molecule in different corneal photocrosslinking procedures are also the subject of this invention.

It is found that that photo-crosslinking effect of the ruthenium compound is concentration-dependent. The concentration of photo-initiator for [Ru(bpy)₃]²⁺ is between of 0.001 M (1 mM) to 0.01 M (10 mM) and SPS is in the range of 0.1 mM to 1 mM. Furthermore, it was discovered that, ruthenium compound or solution at 0.001 M (1 mM) concentration indicates good crosslinking efficiency.

The term “a therapeutically effective amount” of a compound of the present invention refers to a non-toxic and sufficient amount of the compound or solution of the present invention that will elicit the biological or medical response of a subject.

The present invention relates to pharmaceutical compositions including a pharmaceutical carrier and a therapeutically effective amount of ruthenium compound, or a pharmaceutically acceptable salt thereof.

All of the various embodiments of the present invention as disclosed herein relate to methods of treating and/or preventing corneal ectatic disorders as described herein.

Non-limiting examples of corneal ectatic disorders include keratoconus keratoglobus, pellucid marginal degeneration, Terrien's marginal degeneration and post-surgery ectasia.

The present invention relates to the use of ruthenium compound for the preparation of a medicament useful in the treatment and/or prevention of corneal ectatic disorder.

Moreover, the invention relates to a pharmaceutical composition including such compounds (ruthenium (Ru) or Ru/SPS), uses and methods of use for such compounds in the treatment and/or prevention of disorders associated with the corneal ectatic disorders.

These examples are intended to representative of specific embodiments of the invention and are not intended as limiting the scope of the invention.

### Specific Embodiments

In these embodiments, first, the non-lethal concentration of Ruthenium and SPS on the viability of human cell lines was determined. After that, bovine corneas cross-linked with Ruthenium/SPS (Sodium Persulfate) mixture and visible blue light were characterized and compared their biomechanical properties with the conventional crosslinking approach. Our basic goal was to develop an ideal and clinically applicable crosslinking technique that will become the standard therapy for all corneal ectatic pathologies.

### EXAMPLES

### Example 1 Biomechanical Experiments

For biomechanical experiments, bovine corneas were used. First the corneal epithelia were removed with a scalpel blade and 8 mm central corneal buttons were isolated from bovine eyes with a trephine blade (Katena, Parsippany, NJ, USA). After that, corneal buttons separated into three different experimental groups:
Ruthenium (Ru)-Sodium persulfate (SPS)/visible light,
Riboflavin/UV-A light, and
Control groups.

When Ru(bpy)₃, an organoruthenium complex, is mixed with sodium persulfate (SPS) and the mixture is illuminated with visible light, Ru(II) oxidizes into Ru(III) and free electrons are accepted by SPS. Upon electron donation, SPS divides into sulfate anions and radicals and eventually forms new covalent bonds between nearby tyrosine groups on collagen chains. This dityrosine bond generation from tyrosine residues on collagen fibrils could be controlled with visible light and photoinitiator concentrations. This approach is currently used for various tissue engineering methods.

[Ru(bpy)₃]²⁺ and SPS solution was prepared in PBS via dissolving Tris(2,2′-bipyridyl)dichlororuthenium(II) hexahydrate (544981, Sigma-Aldrich, MO, USA) and sodium persulfate (216232, Sigma-Aldrich, MO, USA). Ru-SPS solution was prepared in two different concentrations: 0.001 M (1 mM) and 0.0001 M (0.1 mM) was dropped on corneal buttons and the samples were incubated for 5 min for the samples in the Ru-SPS experimental group. Then all solutions were removed, and 430 nm visible light (3 mW/cm²) was applied to the samples for 40 min. To mimic the standard CXL corneal collagen crosslinking procedure for the riboflavin experimental group, 0.002 M (2 mM) riboflavin 5′-monophosphate solution (18167, Cayman Chemical, MI, USA) was dropped on corneal buttons and the samples were incubated for 5 min. Then riboflavin solution was removed, and 365 nm visible light (3 mW/cm²) applied to the cornea samples for 40 min. No solution or light was applied to the corneas in the control group.

### Example 2 Cell Viability Tests

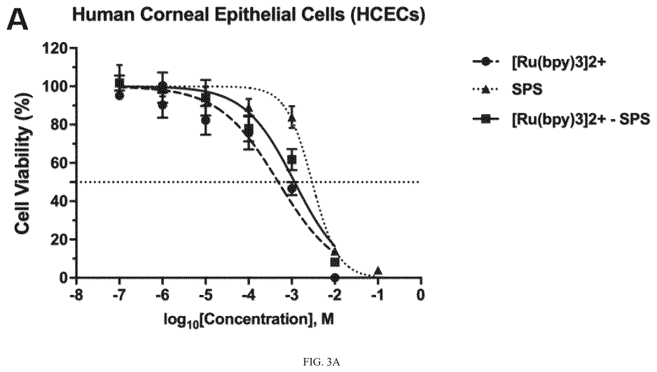

Furthermore, the cytotoxic effects of Ruthenium and SPS compound on human corneal epithelial cells (HCECs) and limbal mesenchymal stem cells (LMSCs) were investigated to determine the ruthenium concentration and time needed for the cross-linking process. The relative viabilities of cells were detected by CellTiter-Glo® Luminescent Cell Viability Assay (Promega, Mannheim, Germany) detected the relative viabilities of cells, which is a method used to detect the number of viable cells depending on the amount of ATP in alive cells. Treatment with Ruthenium or/and SPS exhibited dose-dependent cytotoxicity influence on the viability of cells. The half-maximum inhibitory concentration (IC50) of ruthenium with SPS treatment was found as 1.162 mM for HCEC cells and 2.732 mM for LMSC cells. (FIGS. 3A-3B)

### Example 3 Flow Cytometer Results

The effects of [Ru(bpy)₃]²⁺ and SPS solutions on cell viability and apoptosis were examined by Annexin-V/propidium iodide (PI) staining in flow cytometry. LMSCs and HCECs (10⁵ cells/well) were plated in 6-well culture dishes. [Ru(bpy)₃]²⁺ and SPS in various concentrations (1 mM and 10 mM) were added to the cell culture medium. The cells were incubated for 1 hour at 37 °C. After the treatment with the solutions, the cells were collected and washed with PBS at 4 °C. The cells were resuspended in 1× binding buffer at a 1×105 cells/ml density. 100 μl of the cell suspension were labeled with 2.5 μl of fluorochrome-conjugated Annexin V and 5 μl of PI according to the manufacturer's protocol (BioLegend, San Diego, CA, USA) and analyzed by using a flow cytometer (Attune N. T Flow Cytometer, Thermo Fisher Scientific, Waltham, MA, USA).

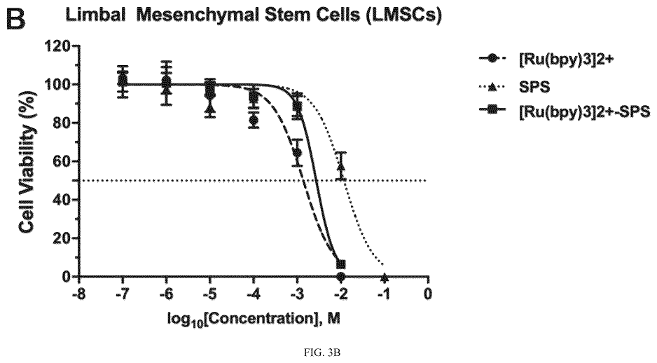

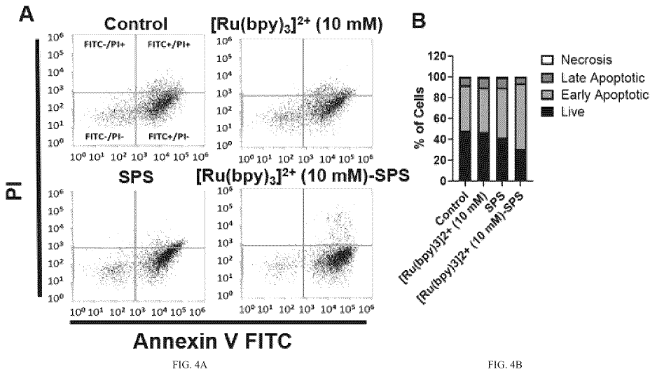

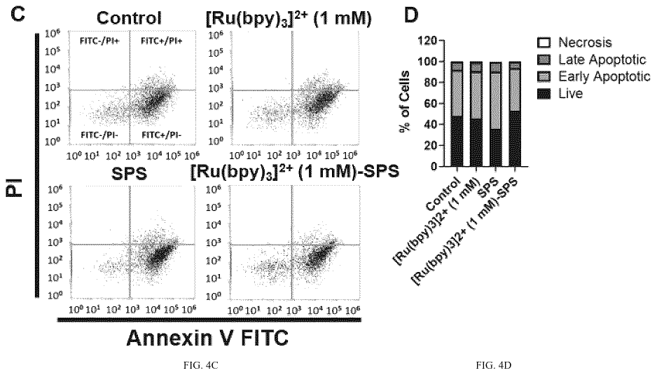

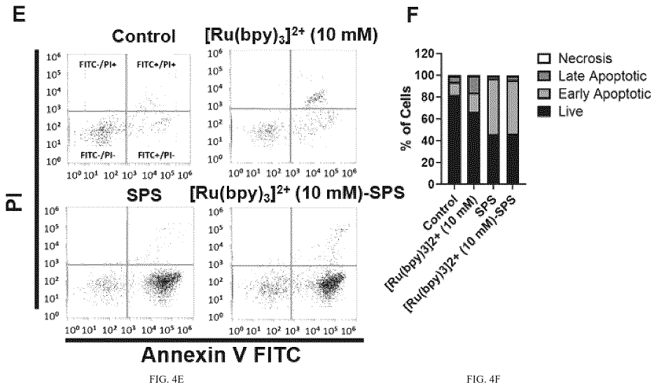

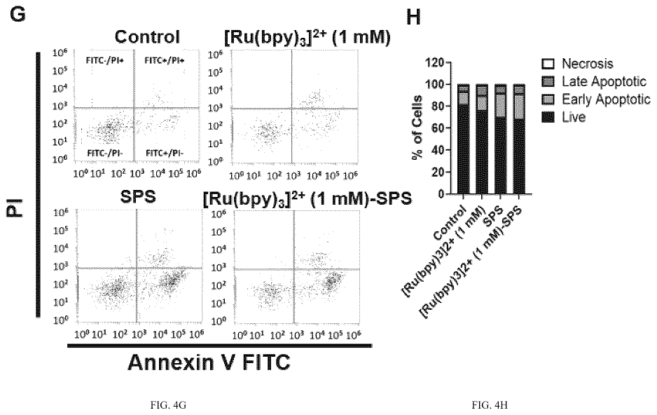

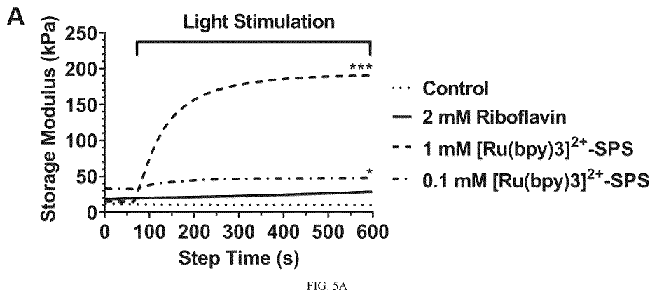

Annexin V-FITC and propidium iodide staining provide sensitive outcomes for detecting cellular apoptosis. While annexin V-FITC binds on the surface of cells that undergo programmed cell death, PI detects necrotic or late apoptotic cells characterized by loss of plasma and nuclear membrane integrity. Cells treated with [Ru(bpy)₃]²⁺ and/or SPS for 1 hour were harvested after treatment and stained with Annexin V-FITC and PI. It was discerned that only SPS treatment displayed a higher degree of cell death than ruthenium-SPS treated cells, which is consistent with the results from the CTG assay (FIGS. 4A-4H).

### Example 4 Rheological Measurements

Rheological measurements were carried out in time sweep mode with 1 Hz frequency, 1% strain using TA Instruments AR/DHR series rheometer (New Castle, DE) equipped with two different light sources: visible light (450 nm) and UV-A light (λ=365 nm). Bovine corneal buttons were placed on a 200 mm quartz plate. The working gap was set according to the thickness of the cornea. Storage and loss moduli profiles were recorded as a function of time continuously for 40 min at ambient conditions. The time required for crosslinking has been estimated through monitoring the storage and loss moduli (G′ and G″).

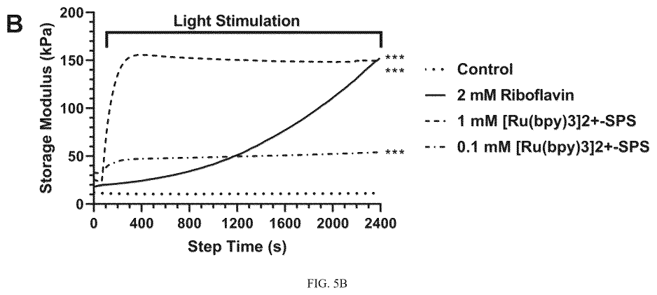

Results shows that 1 mM ruthenium crosslinked corneas achieved 148.53 kPa average storage modulus whereas 2 mM riboflavin crosslinked corneas achieved only 92.88 kPa after 30 min. We also compared different concentrations of ruthenium solutions (0.1 mM vs 1 mM). 0.1 mM ruthenium creates a significantly lower storage modulus increase (51.55 kPa) compared to 1 mM (148.53 kPa). This result indicates that photo-crosslinking effect of the ruthenium compound is concentration-dependent (FIGS. 5A-5B).

### Example 5 Biomechanical Tests

The stress-strain test was performed on a full-thickness minus the epithelium corneal strips dissected from the bovine corneas. The mechanical properties of all the samples were determined using force and displacement measurements. The initial cross-sectional area and the corresponding force measurements of each test sample were used to calculate the engineering stress. The maximum displacement at the breakage point of each sample was used to estimate the change in the length of each sample. This change in the length was related nominally to the initial length of the corresponding test sample to calculate the engineering strain. The modulus of elasticity and the ultimate tensile strength of each sample was then estimated by the engineering stress-strain curves. The ultimate tensile strength was defined as the peak value of stress on the stress-strain curve before the breakage point of the sample when being stretched. The linear regression analysis between the zero point and the point that equals 70% of the maximum stress for each sample was used to compute the modulus of elasticity. The modulus of elasticity is a material-dependent property such that it depends upon the internal structure and the bond strength between the atoms of each sample. Therefore, the modulus of elasticity remained similar for one sample type; however, it differed for other sample types. The average stress versus strain curves for control, riboflavin, and the ruthenium type samples are illustrated in FIG. 6A. Samples treated with ruthenium and visible light have exhibited approximately 24% and 9% higher tensile strength and modulus of elasticity, respectively, than the control and riboflavin treated samples. On the other hand, the ultimate tensile strength and the modulus of elasticity of riboflavin-treated samples were approximately 16% higher than the control samples. The increased modulus of elasticity values for ruthenium and the riboflavin treated samples illustrate an overall increasing stiffness whereas decreasing strain trend compared to the control samples.

Swelling of the corneal stroma affects corneal transparency leading to vision impairments, one of the main reasons for cornea-related vision loss in keratoconus. Therefore, it is vital to understand the hydration behavior of corneas after corneal crosslinking procedures. For the osmotic stress test, crosslinked corneal buttons were incubated in 15% dextran-500 PBS solution overnight to achieve equilibrium. Both ruthenium and riboflavin-based crosslinked corneas have demonstrated less swelling after three hours of incubation in distilled water than normal corneas (FIG. 6B). The results indicate that the swelling behavior significantly decreased after both CXL procedures, which means both CXL procedures have similar crosslinking strength against corneal swelling.

Corneal optical transparencies were measured in corresponding light wavelengths for each experimental group. For the 2 mM riboflavin-based crosslinked corneas, average optical transparency in 365 nm was measured as 0.713±0.222. In the same conditions, the average optical transparency for 1 mM ruthenium-based crosslinked corneas was measured as 0.724±0.050 in 430 nm light wavelength, which is correlated with the riboflavin-based CXL procedure. When these two crosslinking groups were compared, they did not significantly differ in statistical tests (FIG. 6C). According to these results, the ruthenium-based CXL procedure leads to similar optical transmittance levels to the riboflavin-based method, which shows that the ruthenium-based visible light corneal crosslinking procedure will not affect vision during the clinical application. The Lambert-Beer formula was applied for both photoinitiator solutions. While the mean distance that riboflavin traveled in the bovine cornea is 719 μm, the mean distance for ruthenium solution is 575 μm. This result shows that ruthenium will reach fewer toxic levels in the inner parts of the cornea, which enables lower corneal endothelial toxicity for further clinical applications.

In keratoconus, increased collagenolytic activity plays a crucial role in corneal thinning and pathogenesis. Because of this, ophthalmologists try to increase the strength of the cornea against enzymatic stress by corneal CXL procedures.[40] Therefore, it is crucial to study the effect of crosslinking treatment on the resistance of ruthenium-based crosslinked corneas against digestion by collagenases. The samples treated with ruthenium and visible light remained after 24 hours of digestion. However, non-crosslinked control corneas and those treated with riboflavin and UV-A light were digested entirely after 24 hours (FIG. 6D). The increased resistance of the ruthenium and visible light crosslinked cornea to enzymatic digestion can be explained by the collagen fibrils' tertiary structure changes caused by crosslinking, thus preventing proteolytic enzymes from accessing their specialized cleavage sites due to steric hindrance.

### Example 6 UV & Visible Spectrophotometer Results

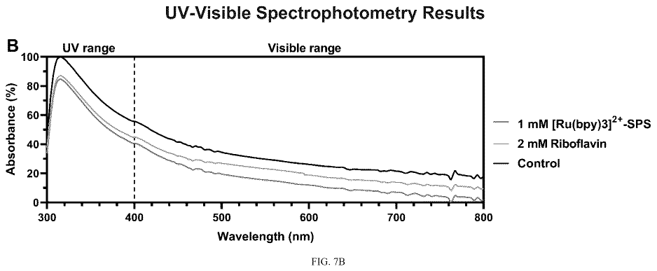

Optical transmission and absorption tests were performed on de-epithelized bovine corneas for the UV and visible light spectrum. Control and crosslinked corneas were submerged in PBS, and their optical transmission was assessed using a UV-Vis. Spectrometer (Edinburgh Instruments Spectrofluorometer FS5). Transmittance spectra (% T) were collected from 300 to 800 nm at 1 nm increments. In UV & Visible spectrophotometer results, no significant optical transmission and absorption changes observed on ruthenium based crosslinked corneas compared to riboflavin induced crosslinked corneas. The results are illustrated in FIGS. 7A-7B.

### REFERENCES

[1]. Tan, D. T., & Por, Y.-M. (2007). Current treatment options for corneal ectasia. Current Opinion in Ophthalmology, 18(4), 279-283.
[2]. Pron, G., Ieraci, L., & Kaulback, K. (2011). Collagen cross-linking using riboflavin and ultraviolet-a for corneal thinning disorders: an evidence-based analysis. Ont Health Technol Assess Ser, 11(5), 1-89.
[3]. McCall, A. S., Kraft, S., Edelhauser, H. F., Kidder, G. W., Lundquist, R. R., Bradshaw, H. E., Dedeic, Z., Dionne, M. J. C., Clement, E. M., & Conrad, G. W. (2010). Mechanisms of Corneal Tissue Cross-linking in Response to Treatment with Topical Riboflavin and Long-Wavelength Ultraviolet Radiation (UVA). Investigative Ophthalmology & Visual Science, 51(1), 129-138.
[4]. Molokhia, S., Muddana, S. K., Hauritz, H., Qiu, Y., Burr, M., Chayet, A., & Ambati, B. K. (2020). IVMED 80 eye drops for treatment of keratoconus in patients-Phase 1/2a. Investigative Ophthalmology & Visual Science, 61(7), 2587-2587.
[5]. Ciolino, S. A. B. (2015). Methods for cross-linking corneal collagen with verteporfin for the treatment of disorders of the eye (US Patent No. WO2015130944A1).
[6]. Cherfan, D., Verter, E. E., Melki, S., Gisel, T. E., Doyle, F. J., Jr., Scarcelli, G., Yun, S. H., Redmond, R. W., & Kochevar, I. E. (2013). Collagen cross-linking using rose bengal and green light to increase corneal stiffness. Invest Ophthalmol Vis Sci, 54(5), 3426-3433.
[7]. Martinez, J. D., Naranjo, A., Amescua, G., Dubovy, S. R., Arboleda, A., Durkee, H., Aguilar, M. C., Flynn, H. W., Miller, D., & Parel, J.-M. (2018). Human Corneal Changes After Rose Bengal Photodynamic Antimicrobial Therapy for Treatment of Fungal Keratitis. Cornea, 37(10), e46-e48.
[8]. Herekar, S. V. (2008). Method for equi-dosed time fractionated pulsed uva irradiation of collagen/riboflavin mixtures for ocular structural augmentation.
[9]. Koller, T., Mrochen, M., & Seiler, T. (2009). Complication and failure rates after corneal crosslinking. Journal of Cataract and Refractive Surgery, 35(8), 1358-1362.
[10]. Chen, X. J., Stojanovic, A., Eidet, J. R., & Utheim, T. P. (2015). Corneal collagen cross-linking (CXL) in thin corneas. Eye and Vision, 2.
[11]. Bagga, B., Pahuja, S., Murthy, S., & Sangwan, V. S. (2012). Endothelial Failure After Collagen Cross-Linking With Riboflavin and UV-A: Case Report With Literature Review. Cornea, 31(10), 1197-1200.
[12]. Sharma, A., Mirchia, K., Mohan, K., Sharma, R., Nottage, J. M., & Nirankari, V. S. (2013). Persistent Corneal Edema After Collagen Cross-Linking for Keratoconus REPLY. American Journal of Ophthalmology, 155(4), 775-776.
[13]. Arora, R., Jain, P., Jain, P., Manudhane, A., & Goyal, J. (2016). Results of Deep Anterior Lamellar Keratoplasty for Advanced Keratoconus in Children Less Than 18 Years REPLY. American Journal of Ophthalmology, 167, 97-98.
[14]. Wollensak, G., Spoerl, E., Wilsch, M., & Seiler, T. (2004). Keratocyte apoptosis after corneal collagen cross-linking using riboflavin/UVA treatment. Cornea, 23(1), 43-49.
[15]. K. Kalyanasundaram, Photophysics, photochemistry and solar energy conversion with tris(bipyridyl)ruthenium(II) and its analogues, Coord. Chem. Rev. 46(1982 ) 159-244. doi.org/10.1016/0010-8545(82)85003-0.
[16]. H. P. Iseli, M. Popp, T. Seiler, E. Spoerl, M. Mrochen, Laboratory measurement of the absorption coefficient of riboflavin for ultraviolet light (365 nm), J. Refract. Surg. 27 (2011) 195-201. doi.org/10.3928/1081597X-20100604-01.

What is claimed is:

1. A method for a photo-crosslinking of a corneal collagen under a visible light, comprising:
applying an analgesic solution to a human eye surface and removing an epithelium layer,
applying a photoinitiator pharmaceutical composition comprising a ruthenium compound on a central corneal surface and waiting for 5 min for an absorption, and
exposing a cornea to 3 mW/cm² of the visible light to induce a crosslinking for 40 min.

2. The method according to claim 1, wherein the visible light has a wavelength in a range of 430-450 nm.

3. The method according to claim 2, wherein the wavelength of the visible light is 430 nm or 450 nm.

4. The method according to claim 3, wherein the photoinitiator pharmaceutical composition is a photo-initiator comprising the ruthenium compound and sodium persulfate (SPS).

5. The method according to claim 4, wherein the photoinitiator pharmaceutical composition is an ophthalmic solution.

6. The method according to claim 5, wherein a concentration of the ruthenium compound of the photoinitiator pharmaceutical composition is in a range of 0.001 M to 0.01 M.

7. The method according to claim 5, wherein the photoinitiator pharmaceutical composition comprises 0.001 M of the ruthenium compound and 0.0001 M of the SPS.

8. The method according to claim 6, wherein the method is for a treatment and/or a prevention of a corneal ectatic disorder.

9. A photo-initiator pharmaceutical composition for use in a treatment and/or a prevention of a corneal ectatic disorder, comprising:
a therapeutically effective amount of a ruthenium compound and SPS; and
a pharmaceutically acceptable carrier.

10. The photo-initiator pharmaceutical composition according to claim 9, wherein a concentration of the ruthenium compound is in a range of 0.001 M to 0.01 M.

11. The photo-initiator pharmaceutical composition according to claim 10, wherein the concentration of the ruthenium compound is 0.001 M and a concentration of the SPS is 0.0001 M.

12. The photo-initiator pharmaceutical composition according to claim 11, wherein the photo-initiator pharmaceutical composition is an ophthalmic solution.

13. The photo-initiator pharmaceutical composition according to claim 12, wherein the photo-initiator pharmaceutical composition is an eye drop.

14. A ruthenium compound for use in a treatment and/or a prevention of a corneal ectatic disorder.
